(19) United States
(12) Patent Application Publication
(10) Pub. No.: US 2023/0226333 A1
(43) Pub. Date: Jul. 20, 2023

(54) APPLICATOR, MICRO NEEDLE PATCH KIT, AND INSERTION METHOD

(71) Applicant: MEDRx Co., Ltd., Higashikagawa-shi, Kagawa (JP)

(72) Inventors: Katsunori KOBAYASHI, Higashikagawa-shi, Kagawa (JP); Hidetoshi HAMAMOTO, Higashikagawa-shi, Kagawa (JP)

(21) Appl. No.: 18/009,634

(22) PCT Filed: Jun. 10, 2021
(86) PCT No.: PCT/JP2021/022133

§ 371 Date: Dec. 9, 2022

(30) Foreign Application Priority Data
Jun. 12, 2020 (JP) 2020102540

(51) Int. Cl. A61M 37/00

(52) U.S. Cl. CPC A61M 37/0015; A61M 2037/0023; A61M 2037/0061

(57) ABSTRACT

An applicator for inserting a micro needle array (4) into skin includes a holding part (211) that holds the micro needle array (4), and an insertion assist part (223) that forms an insertion complete state in which a central array region of the micro needle array (4) further protrudes on the skin side than a peripheral array region provided closer to a peripheral side of the micro needle array (4) than the central array region.

### CROSS-REFERENCE TO RELATED APPLICATION

This application is based upon and claims the benefit of priority from Japanese Patent Application No. 2020-102540, filed on Jun. 12, 2020, the entire contents of which are incorporated herein by reference.

### BACKGROUND OF THE INVENTION

### Field of the Invention

The present disclosure relates to an applicator for inserting a micro needle array into skin, a micro needle patch kit including the applicator, and an insertion method of inserting a micro needle array into skin.

### Description of the Related Art

Recently, a drug administration system using a micro needle array has been attracting attention as one of transdermal-absorption drug administration systems. In the micro needle array, fine needles having lengths of several hundred microns are highly densely formed on a base material, and the needles on the surface or inside of which a target drug (molecules of vaccine, protein, peptide, or the like) is supported are inserted into skin so that the drug is directly transported to inner skin or top skin. Drug administration using the micro needle array has a number of advantages that, for example, the drug administration does not strain the liver unlike oral medicine, the drug administration causes no pain along with needle insertion unlike an injected drug, and the drug administration can reduce an adverse effect due to temporary excessive drug absorption.

A micro needle array disclosed in Japanese Patent No. 6546091 below is intended to homogenously increase wrinkles of skin and effectively insert micro needles into the skin at which concave and convex parts are formed due to the wrinkles.

Japanese Patent No. 6546091 is intended to effectively insert micro needles into skin at which wrinkles are formed but does not disclose reliable insertion of a large number of micro needles provided at the micro needle array into skin irrespective of the existence of wrinkles.

The present disclosure is intended to provide an applicator capable of reliably inserting a large number of micro needles provided at a micro needle array into skin, a micro needle patch kit including the applicator, and an insertion method of reliably inserting a micro needle array into skin.

### SUMMARY OF THE INVENTION

To solve the above-described problem, the inventors focused on the way of force application to a micro needle array. The inventors have found that, when force toward skin is applied to the entire micro needle array, micro needles provided in a region on a central side of the micro needle array are not sufficiently inserted into skin in some cases. The present disclosure is based on this novel knowledge.

The present disclosure is an applicator for inserting a micro needle array into skin and includes a holding part that holds the micro needle array, and an insertion assist part that forms an insertion complete state in which a central array region of the micro needle array further protrudes on the skin side than a peripheral array region provided closer to a peripheral side of the micro needle array than the central array region.

As the micro needle array is pressed into skin, the skin becomes dented due to elasticity thereof in accordance with force that presses the skin. The pressing is performed so that the central array region further protrudes on the skin side than the peripheral array region along the dented skin, and accordingly, micro needles are inserted into the skin by equally receiving the force. To achieve this inserted state, the holding part holds the micro needle array and the insertion assist part forms the insertion complete state in which the central array region further protrudes on the skin side than the peripheral array region, and thus, not only micro needles provided in the peripheral array region but also micro needles provided in the central array region can be reliably inserted into the skin.

In the present disclosure, the insertion assist part may form a pre-insertion state in which the central array region does not further protrude on the skin side than the peripheral array region before the insertion complete state.

In the pre-insertion state, no concave and convex parts are formed at the micro needle array, and the micro needles provided in the peripheral array region and the micro needles provided in the central array region can contact the skin. In a state in which no force toward the skin is applied, the central array region does not further protrude on the skin side than the peripheral array region, and thus the micro needle array can be held without a load. The micro needle array can be pressed toward the skin side in the pre-insertion state as well, and in this case, the micro needles provided in the peripheral array region and the micro needles provided in the central array region can be inserted into the skin. Thus, it is possible to achieve both capability of holding the micro needle array at the applicator and capability of inserting the micro needle array when force toward skin is applied.

In the present disclosure, when receiving force toward the skin, the insertion assist part may transition from the pre-insertion state and form the insertion complete state.

Since the insertion complete state is formed after the pre-insertion state, micro needles contacting but not completely inserted into the skin can be reliably inserted into the skin by pressing while the central array region further protrudes on the skin side. When force toward skin is applied, the pre-insertion state is formed and then transition is made to the insertion complete state, and thus a process from a state in which micro needles are incompletely inserted into the skin in the pre-insertion state to a state in which the micro needles are completely inserted into the skin in the insertion complete state can be performed through a series of operations.

In the present disclosure, the holding part may be provided on the peripheral array region side of the central array region.

Since the holding part is provided on the peripheral array region side of the central array region, the holding part can be prevented from encumbering motion of the insertion assist part, which enables smooth transition from the pre-insertion state to the insertion complete state.

The present disclosure may further include a first part at which the insertion assist part is provided and a second part at which the holding part is integrally provided at least in the pre-insertion state, and a relative positional relation between the first part and the second part is changeable.

Since the first part at which the insertion assist part is provided can change the positional relation with the second part at which the holding part is integrally provided in the pre-insertion state, the insertion assist part can protrude on the skin side from the second part, thereby forming the insertion complete state.

In the present disclosure, a through-hole may be provided at the second part, and the insertion assist part may be held through the through-hole.

Since the insertion assist part is held through the through-hole, the through-hole can guide the insertion assist part when the first part changes the relative positional relation with the second part, and accordingly, the insertion assist part can stably protrude on the skin side.

In the present disclosure, the through-hole may include a large-diameter part and a small-diameter part, the large-diameter part being provided on the micro needle array side, the small-diameter part being provided at a position farther separated from the micro needle array than the large-diameter part, having a smaller diameter than the large-diameter part, and slidably holding the insertion assist part, a protrusion may be provided at the insertion assist part, and the protrusion may pass through the small-diameter part and generate notification sound when a magnitude of force toward the skin exceeds a predetermined magnitude.

With the generation of the notification sound, it is possible to sense a state in which the magnitude of the force toward the skin exceeds the predetermined magnitude and micro needles are reliably inserted into the skin. The generation of the notification sound serves as a trigger for separation of the applicator from the skin.

In the present disclosure, the through-hole may penetrate through the holding part, and the insertion assist part may further protrude on the skin side than the holding part in the insertion complete state.

Since the through-hole penetrates through the holding part, the risk that the holding part encumbers motion of the insertion assist part is reduced. The holding part maintains integration with the second part and does not move toward the skin side when the insertion assist part further protrudes on the skin side, and accordingly, the insertion assist part presses the micro needle array toward the skin side so that the micro needle array is separated from the holding part. Thus, it is possible to achieve both capability of holding the micro needle array at the applicator and capability of inserting the micro needle array when force toward skin is applied.

In the present disclosure, the holding part may be formed such that a central holding region further protrudes on the skin side than a peripheral holding region provided on the peripheral side of the central holding region, and the insertion assist part may be formed when the central array region is held in the central holding region.

Since the central array region is held in the central holding region, the pre-insertion state in which the central array region does not further protrude on the skin side than the peripheral array region can be formed before the insertion complete state irrespective of the shape of the holding part. When force is further applied while the holding part is pressed against the skin, the micro needle array is deformed along the shape of the holding part and the insertion complete state in which the central array region further protrudes on the skin side than the peripheral array region is formed. Thus, the holding part and the insertion assist part do not need to be provided at different positions but the insertion assist part can be formed with a simple contrivance of configuration, and accordingly, the configuration of the applicator can be simplified.

In the present disclosure, the insertion assist part may be formed such that a central assist region further protrudes on the skin side than a periphery assist region provided on the peripheral side of the central assist region.

Since the insertion assist part has a convex shape in which the periphery assist region is further retracted from the skin side than the central assist region, a part of the micro needle array pressed by the central assist region becomes closer to the skin side and a part of the micro needle array pressed by the periphery assist region relatively becomes closer to the holding part as the insertion assist part applies force toward the skin side to the micro needle array. Accordingly, the micro needle array has a curved shape, which increases capability of inserting the micro needle array and ensures capability of holding the micro needle array.

In the present disclosure, the holding part is formed so that the central holding region further protrudes on the skin side than the peripheral holding region provided on the peripheral side of the central holding region, and the insertion assist part may be formed when the micro needle array is held in the central holding region and the peripheral holding region.

Since the micro needle array is held in the central holding region and the peripheral holding region, a state in which the central array region further protrudes on the skin side along the shape of the holding part than the peripheral array region can be formed and the insertion complete state can be reliably formed.

In the present disclosure, the insertion assist part may form the insertion complete state when receiving force along the skin.

In the present disclosure, the insertion assist part may form the insertion complete state when receiving force that moves closer a pair of facing end parts of the micro needle array.

The present disclosure is a micro needle patch kit including the above-described applicator and a micro needle patch, and in the micro needle patch, the micro needle array is bonded to one surface of a bonding tape.

In the micro needle patch kit of the present disclosure, the bonding tape is provided with, in a region in which the insertion assist part contacts a surface to which the micro needle array is not bonded, a recessed part or surface fabrication that increases friction.

The present disclosure is an insertion method of inserting a micro needle array held by an applicator into skin and includes inserting micro needles into the skin in a state in which a central array region of the micro needle array further protrudes on the skin side than a peripheral array region provided closer to a peripheral side of the micro needle array than the central array region, and removing the micro needle array from the applicator.

According to the present disclosure, it is possible to provide an applicator capable of reliably inserting a large number of micro needles provided at a micro needle array into skin, a micro needle patch kit including the applicator, and an insertion method of reliably inserting a micro needle array into skin.

### BRIEF DESCRIPTION OF THE DRAWINGS

FIG. 1 is an external perspective view illustrating appearance of an applicator in the present embodiment;

FIG. 2 is a back-surface perspective diagram illustrating the applicator illustrated in FIG. 1 when viewed from a back side;

FIG. 3 is a cross-sectional view illustrating a section at a first part and a second part of the applicator illustrated in FIGS. 1 and 2;

FIG. 4 is a cross-sectional view illustrating the section at the first part and the second part of the applicator illustrated in FIGS. 1 and 2;

FIG. 5 is a diagram for description of a situation in which a micro needle array is inserted into skin by using a modification of the applicator illustrated in FIGS. 3 and 4;

FIG. 6 is a diagram for description of a situation in which the micro needle array is inserted into the skin by using the modification of the applicator illustrated in FIGS. 3 and 4;

FIG. 7 is a diagram for description of a situation in which the micro needle array is inserted into the skin by using the modification of the applicator illustrated in FIGS. 3 and 4;

FIG. 8 is a diagram for description of a situation in which the micro needle array is inserted into the skin by using the modification of the applicator illustrated in FIGS. 3 and 4;

FIG. 9 is a diagram for description of a situation in which the micro needle array is inserted into the skin by using the modification of the applicator illustrated in FIGS. 3 and 4;

FIG. 10 is a diagram for description of a modification of an insertion assist part;

FIG. 11 is a diagram for description of another modification of the insertion assist part;

FIG. 12 is a diagram for description of another modification of the insertion assist part;

FIG. 13 is a diagram for description of another modification of the insertion assist part;

FIG. 14 is a diagram for description of the modification of the insertion assist part;

FIG. 15 are diagrams for description of another modification of the insertion assist part;

FIG. 16 is a diagram for description of a central array region and a peripheral array region of the micro needle array;

FIG. 17 is a diagram for description of another example of the central array region and the peripheral array region of the micro needle array;

FIG. 18 is a diagram for description of another example of the central array region and the peripheral array region of the micro needle array;

FIG. 19 are diagrams for description of another modification of the insertion assist part; and

FIG. 20 is a diagram for description of another modification of the insertion assist part.

### DETAILED DESCRIPTION OF THE PREFERRED EMBODIMENTS

The present embodiment will be described below with reference to the accompanying drawings. To facilitate understanding of the description, identical constituent components in the drawings are denoted by the same reference sign when possible, and duplicate description thereof is omitted.

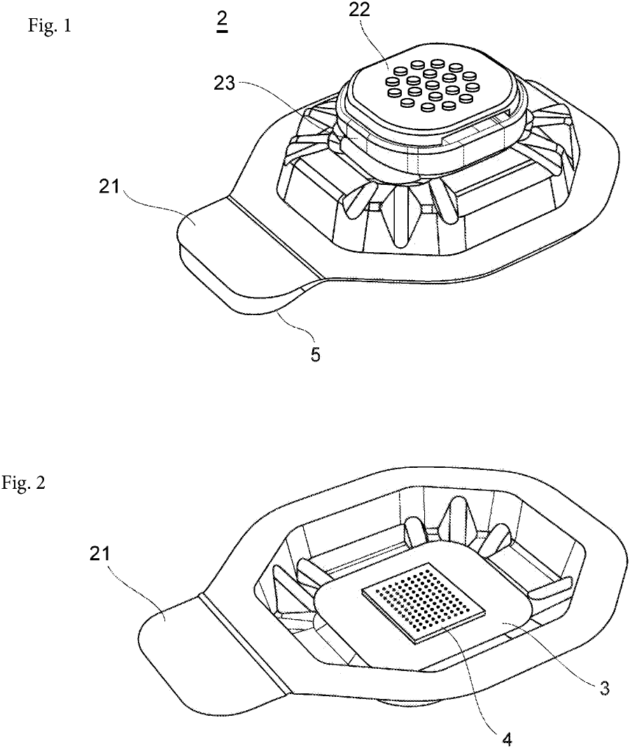

As illustrated in FIG. 1, an applicator 2 in the present embodiment includes a housing 21, a first part 22, and a second part 23. The housing 21 is a dome-shaped member in which an opened space is provided. The housing 21 is shaped by a well-known method of shaping a thin resin sheet or film or a well-known method of injecting and shaping melted resin in the cavity of a mold.

A material that is suitable for shaping of the housing 21 is preferably a material that is flexible and easily bendable, deformable, and restorable, and examples thereof include resins such as polyethylene, polypropylene, polyethylene terephthalate, polystyrene, nylon, acrylic, silicone, and ABS. The shape of the housing 21 and the thickness of each part thereof are determined as appropriate such that desired deformation occurs when a person presses the housing 21 from above with a finger. The shape of the housing 21 may be provided with a valley-folding part and easily bendable at the valley-folding part. The thickness of the housing 21 is, for example, 50 µm approximately to 1 mm, or 100 µm to 500 µm.

As illustrated in FIG. 2, a bonding tape 3 and a micro needle array 4 are disposed inside the housing 21. The housing 21 may be used as a container with its opening part sealed by a protection sheet 5 after the bonding tape 3 and the micro needle array 4 are disposed. In the micro needle array 4, needles having lengths of several hundred microns are highly densely formed on a base material, and a target drug (molecules of vaccine, protein, peptide, low-molecular compound, or the like) is supported on the surface of each needle or inside thereof. When the micro needle array 4 is bonded to skin, the needles of the micro needle array 4 are inserted into skin and the drug is directly transported to inner skin or top skin.

The bonding tape 3 is an adhesive tape for inserting and fixing the micro needle array 4 into skin. The micro needle array 4 is bonded to the bonding tape 3. The bonding tape 3 is bonded at a position on the inner surface of the housing 21, the position corresponding to the first part 22 and the second part 23. The bonding tape 3 may be made of a material having a moderately high stiffness to prevent the micro needles from being displaced upward due to elasticity of skin, and may have a thickness and a shape with which the stiffness can be ensured. When the bonding tape 3 has a moderately high stiffness, the bonding tape 3 maintains a plane shape after bonding, and the micro needles are held in a pressurized state in skin, which improves capability of inserting the micro needles.

The applicator 2 is disposed on skin such that the micro needle array 4 faces the skin side. Thereafter, the first part 22 is pressed toward the skin side so that the micro needle array 4 and the bonding tape 3 are transferred onto the skin.

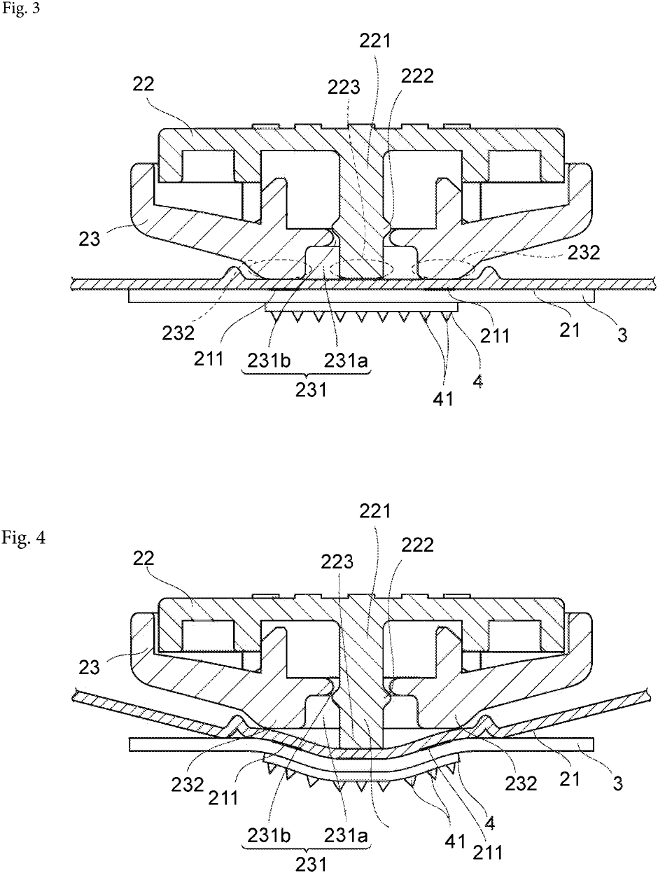

FIG. 3 illustrates a section at the first part 22 and the second part 23 in a pre-insertion state. In the pre-insertion state, the applicator 2 is disposed such that the micro needle array 4 contacts skin. The micro needle array 4 is provided with micro needles 41 at which the target drug is supported. In the pre-insertion state, some of the micro needles 41 may be inserted into skin. In the present embodiment, a surface of the micro needle array 4 on which the micro needles 41 are formed is referred to as a principal surface and a surface opposite the principal surface is referred to as a back surface in the following description.

A through-hole 231 is provided at the second part 23. The through-hole 231 corresponds to a position in the housing 21 at which the micro needle array 4 is bonded. The through-hole 231 includes a large-diameter part 231a and a small-diameter part 231b. The large-diameter part 231a has a larger diameter than the small-diameter part 231b and is provided on the micro needle array 4 side thereof. The small-diameter part 231b is provided at a position further separated from the micro needle array 4 than the large-diameter part 231a and has a smaller diameter than the large-diameter part 231a. The small-diameter part 231b slidably holds a pressing rod 221 of the first part 22.

The first part 22 is provided on a side opposite the housing 21 with respect to the second part 23. The first part 22 includes the pressing rod 221. The pressing rod 221 penetrates through the small-diameter part 231b of the second part 23. The pressing rod 221 is provided with a protrusion 222. As illustrated in FIG. 3, in a state before the first part 22 is pressed toward the skin side, the protrusion 222 is positioned on a side opposite the micro needle array 4 with respect to the small-diameter part 231b. When the magnitude of force applied toward skin S at the first part 22 exceeds a predetermined magnitude, the protrusion 222 provided at the first part 22 passes through the small-diameter part 231b and generates notification sound.

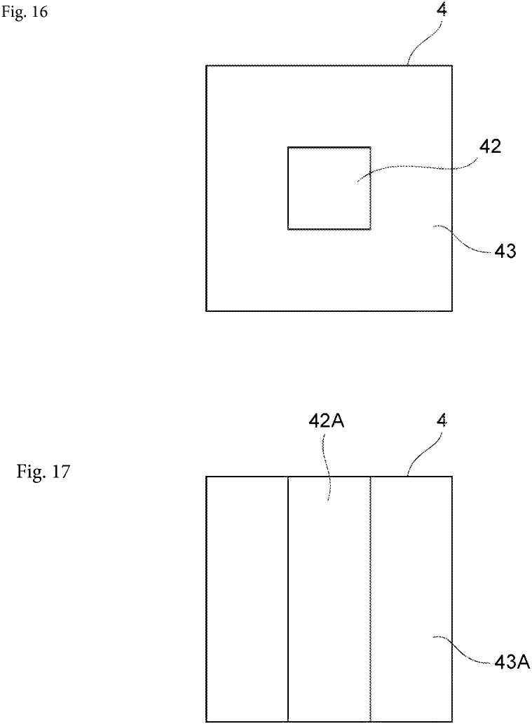

An insertion assist part 223 is provided at a part of the pressing rod 221 contacting the housing 21. The insertion assist part 223 is disposed in contact with the back surface of the micro needle array 4. The insertion assist part 223 is a part that causes a central array region as part of the micro needle array 4 to protrude on the skin side, thereby forming an insertion complete state. The central array region will be described below with reference to FIGS. 16 to 18.

As illustrated in FIG. 16, a central array region 42 and a peripheral array region 43 are provided at the micro needle array 4. The central array region 42 is a region including the barycenter of the micro needle array 4 and provided on the inner side of the peripheral array region 43 in an optional direction along the principal surface on which the micro needles 41 are provided. The peripheral array region 43 is a region including any part of the outer periphery of the micro needle array 4.

The insertion assist part 223 forms the insertion complete state in which the central array region 42 further protrudes on the skin side than the peripheral array region 43. The insertion assist part 223 contacts at least part of the central array region 42 with the housing 21 interposed therebetween. The insertion assist part 223 does not necessarily need to have the same shape as the central array region 42 but may have a shape such as a cylindrical shape having a smaller contact surface than the central array region 42.

In the example illustrated in FIG. 16, the central array region 42 is provided on the inner side of the peripheral array region 43 in all directions passing through the barycenter of the micro needle array 4. The central array region does not necessarily need to be provided on the inner side of the peripheral array region 43 in all directions passing through the barycenter of the micro needle array 4. Such an example will be described below with reference to FIG. 17.

As illustrated in FIG. 17, a central array region 42A of the micro needle array 4 includes the barycenter of the micro needle array and is provided on the inner side of a peripheral array region 43A in some directions passing through the principal surface of the micro needle array 4. Part of the central array region 42A includes the outer periphery of the micro needle array 4.

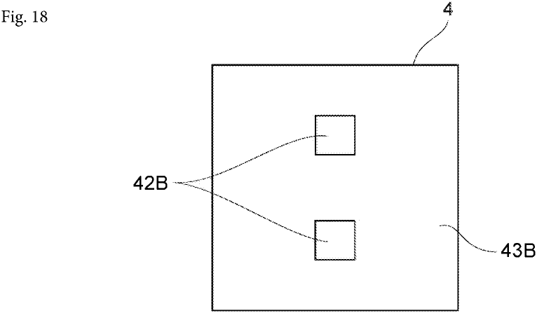

In the example illustrated in FIGS. 16 and 17, the central array regions 42 and 42A are each formed as a single region. Each central array region does not necessarily need to be a single region but may be formed as a plurality of regions. Such an example will be described below with reference to FIG. 18.

As illustrated in FIG. 18, two central array regions 42B are provided in the micro needle array 4. The central array regions 42B are provided on the inner side of a peripheral array region 43B in all directions passing through the principal surface of the micro needle array 4.

The central array regions 42, 42A, and 42B and the peripheral array regions 43, 43A, and 43B described above with reference to FIGS. 16, 17, and 18 are merely exemplary and may be provided in any form with which the insertion assist part can contact the central array region and cause the central array region to protrude on the skin side.

Description continues with reference to FIGS. 3 and 4 again. When force is applied to the first part 22 in the state illustrated in FIG. 3, force is applied to the housing 21 by an insertion support part 232 of the second part 23 while the protrusion 222 does not pass through the small-diameter part 231b yet. The insertion support part 232 is provided to determine the outer periphery of the large-diameter part 231a. Although not explicitly illustrated in FIG. 3, the housing 21 and the micro needle array 4 are each deformed in a trapezoid shape along an outside part of the insertion support part 232 when force is applied to the first part 22 and the second part 23.

A state illustrated in FIG. 4 is obtained when the first part 22 is further pressed toward the skin side in the state illustrated in FIG. 3. As the first part 22 is pressed toward the skin side, the pressing rod 221 penetrates through the through-hole 231 and protrudes on the micro needle array 4 side. The protrusion 222 provided at the pressing rod 221 passes through the small-diameter part 231b and is positioned on the large-diameter part 231a side.

As illustrated in FIG. 4, when force toward the skin is applied to the first part 22, the insertion assist part 223 provided at the leading end of the pressing rod 221 generates force that presses the micro needle array 4 toward the skin through the housing 21 and the bonding tape 3. The housing 21 and the micro needle array 4 each being deformed in the trapezoid shape by the force applied from the insertion support part 232 further receive the force from the insertion assist part 223 and are curved as a whole.

FIG. 4 illustrates the section at the first part 22 and the second part 23 in the insertion complete state. In the insertion complete state, the central array regions 42, 42A, and 42B (refer to FIGS. 16, 17, and 18) of the micro needle array 4 further protrude on the skin side than the peripheral array regions 43, 43A, and 43B (refer to FIGS. 16, 17, and 18). In the insertion complete state, the micro needle array 4 and the bonding tape 3 are transferred onto the skin and most (preferably all) of the micro needles 41 are inserted into the skin.

In the example described above with reference to FIGS. 3 and 4, the micro needle array 4 is held in the housing 21 by a holding part 211 with the bonding tape 3 interposed therebetween. The holding part 211 is made of a two-sided adhesive tape. The holding part 211 is provided at a position corresponding to the insertion support part 232 of the second part 23.

When the first part 22 and the second part 23 are separated from the skin together with the housing 21 after the insertion complete state illustrated in FIG. 4, holding by the holding part 211 is canceled and the micro needle array 4 and the bonding tape 3 remain on the skin.

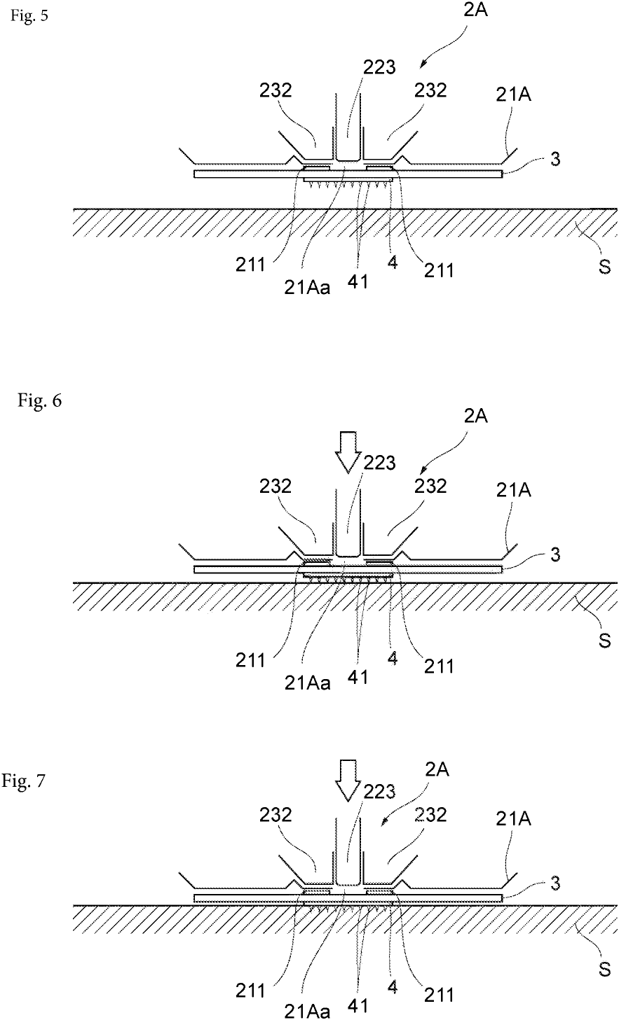

An applicator 2A as a modification will be described below with reference to FIGS. 5, 6, 7, 8, and 9. FIG. 5 illustrates a state in which the applicator 2A is placed on the skin S. In the state illustrated in FIG. 5, the micro needle array 4 and the skin S have a gap of several millimeters approximately therebetween.

The applicator 2A includes a housing 21A in place of the housing 21 of the applicator 2. The housing 21A is provided with a hole 21Aa at a position corresponding to the insertion assist part 223. The holding part 211 for holding the micro needle array 4 and the bonding tape 3 is provided at a part of the housing 21 corresponding to the micro needle array 4. The holding part 211 is made of a two-sided adhesive tape. The holding part 211 is provided at a position corresponding to the insertion support part 232 of the second part 23.

FIG. 6 illustrates a state in which force is applied in an illustrated arrow direction that is a direction toward the skin S after the applicator 2 is placed on the skin S. A large number of micro needles 41 are provided at the micro needle array 4 and contact the skin S.

A state illustrated in FIG. 7 is obtained when force is further applied in the illustrated arrow direction as the direction toward the skin S in the state illustrated in FIG. 6. In the state illustrated in FIG. 7, at least some of the micro needles 41 are inserted into the skin S. The states of the applicator 2 illustrated in FIGS. 5, 6, and 7 are the pre-insertion state. In the present embodiment, the insertion support part 232 presses the micro needles 41 toward the skin S ahead of the other part, and thus mainly the micro needles 41 provided at a position corresponding to the insertion support part 232 are inserted into the skin S.

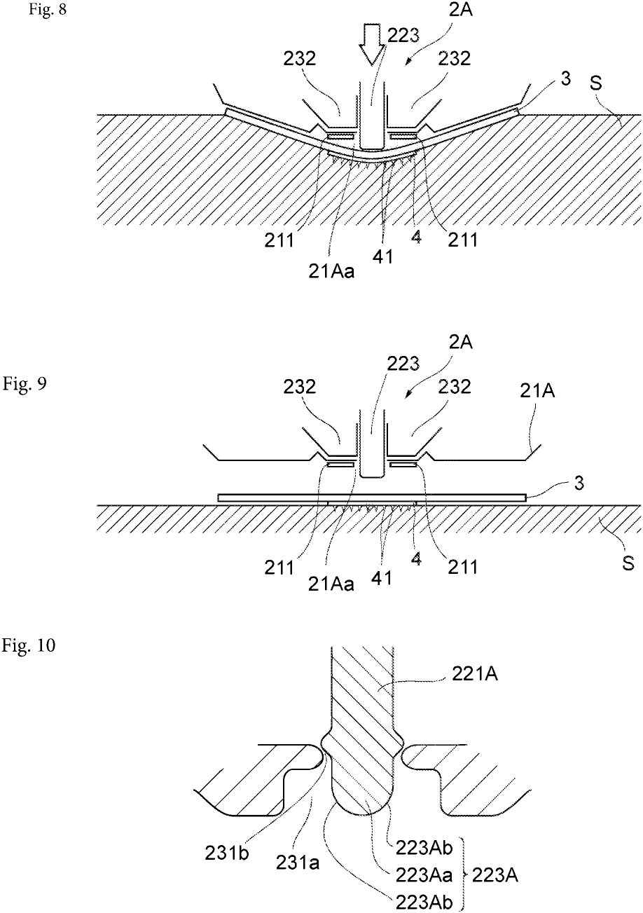

A state illustrated in FIG. 8 is obtained when force is further applied in the arrow direction toward the skin S in the state illustrated in FIG. 7. FIG. 8 illustrates the insertion complete state. As illustrated in FIG. 8, the insertion assist part 223 protrudes relative to the insertion support part 232. The state in which the insertion support part 232 applies force ahead of the other part transitions to a state in which the insertion assist part 223 applies force to the micro needle array 4. The insertion assist part 223 penetrates through the hole 21Aa provided at the housing 21 and causes the central array regions 42, 42A, and 42B (refer to FIGS. 16, 17, and 18) of the micro needle array 4 to further protrude on the skin S side than the peripheral array regions 43, 43A, and 43B (refer to FIGS. 16, 17, and 18).

Since the insertion assist part 223 penetrates through the hole 21Aa, the insertion assist part 223 applies no force to a part at which the holding part 211 is provided. Accordingly, the holding part 211 maintains the state of contacting the insertion support part 232, and the bonding tape 3 is separated from the holding part 211.

The micro needle array 4 is more deeply pressed into the skin S at a part corresponding to the insertion assist part 223 and is curved as a whole. In this state, the large number of micro needles 41 provided at micro needle array 4 are uniformly inserted into the skin S.

A state illustrated in FIG. 9 is obtained when force toward the skin S is canceled in the state illustrated in FIG. 8. In the state illustrated in FIG. 9, the insertion assist part 223 and the insertion support part 232 are separated from the micro needle array 4, and the skin S and the micro needle array 4 recover to their unloaded states. The micro needle array 4 maintains the state in which the large number of micro needles 41 are inserted into the skin S, and is fixed to the skin S by the bonding tape 3.

In the above description, the insertion support part 232 applies force that presses the micro needles 41 against the skin S ahead of the insertion assist part 223, but the insertion assist part 223 may apply force ahead of the other part. Alternatively, only the insertion assist part 223 may apply force that presses the micro needles 41 against the skin S. When the insertion assist part 223 applies force that presses the micro needles 41 against the skin S ahead of the other part or by itself, the insertion assist part 223 may protrude after the micro needles 41 approach the skin S to some extent.

As described above, the applicators 2 and 2A in the present embodiment are each an applicator for inserting the micro needle array 4 into the skin S. The applicators 2 and 2A each include the holding part 211 that holds the micro needle array, and the insertion assist part 223 that forms the insertion complete state in which the corresponding one of the central array regions 42, 42A, and 42B of the micro needle array further protrudes on the skin side than the corresponding one of the peripheral array regions 43, 43A, and 43B further provided on the peripheral side of the micro needle array than the central array region.

As the micro needle array 4 is pressed against the skin S, the skin S becomes dented in accordance with force pressing the skin S due to elasticity of the skin S. The micro needle array 4 is pressed along the dented skin S such that the central array regions 42, 42A, and 42B further protrude on the skin S side than the peripheral array regions 43, 43A, and 43B, and accordingly, the micro needles 41 equally receive force and are inserted into the skin S. To achieve this inserted state, the holding part 211 holds the micro needle array 4 and the insertion assist part 223 forms the insertion complete state in which the central array regions 42, 42A, and 42B further protrude on the skin S side than the peripheral array regions 43, 43A, and 43B. Accordingly, not only the micro needles 41 provided in the peripheral array regions 43, 43A, and 43B but also the micro needles 41 provided in the central array regions 42, 42A, and 42B can be reliably inserted into the skin.

In the present embodiment, the insertion assist part 223 forms, before the insertion complete state, the pre-insertion state in which the central array regions 42, 42A, and 42B do not further protrude on the skin side than the peripheral array regions 43, 43A, and 43B.

In the pre-insertion state, no concave and convex parts are formed at the micro needle array 4, and the micro needles 41 provided in the peripheral array regions 43, 43A, and 43B and the micro needles 41 provided in the central array regions 42, 42A, and 42B can contact the skin S. In a state in which no force toward the skin S is applied, the central array regions 42, 42A, and 42B do not further protrude on the skin S side than the peripheral array regions 43, 43A, and 43B, and thus the micro needle array 4 can be held without a load. The micro needle array 4 can be pressed toward the skin S side in the pre-insertion state as well, and in this case, the micro needles 41 provided in the peripheral array regions 43, 43A, and 43B and the micro needles 41 provided in the central array regions 42, 42A, and 42B can be inserted into the skin S. Thus, it is possible to achieve both capability of holding the micro needle array 4 at the applicators 2 and 2A and capability of inserting the micro needle array 4 when force toward the skin S is applied.

In the present embodiment, when receiving force toward the skin S, the insertion assist part 223 transitions from the pre-insertion state and forms the insertion complete state.

Since the insertion complete state is formed after the pre-insertion state, the micro needles 41 contacting but not completely inserted into the skin S can be reliably inserted into the skin S by pressing while the central array regions 42, 42A, and 42B further protrude on the skin S side. When force toward the skin S is applied, the pre-insertion state is formed and then transition is made to the insertion complete state, and thus a process from a state in which the micro needles 41 are incompletely inserted into the skin S in the pre-insertion state to a state in which the micro needles 41 are completely inserted into the skin S in the insertion complete state can be performed through a series of operations.

In the present embodiment, the holding part 211 is provided on the peripheral array region 43, 43A, and 43B sides of the central array regions 42, 42A, and 42B.

Since the holding part 211 is provided on the peripheral array region 43, 43A, and 43B sides of the central array regions 42, 42A, and 42B, the holding part 211 can be prevented from encumbering motion of the insertion assist part 223, which enables smooth transition from the pre-insertion state to the insertion complete state.

In the present embodiment, the applicators 2 and 2A each include the first part 22 at which the insertion assist part 223 is provided and the second part 23 at which the holding part 211 is integrally provided at least in the pre-insertion state, and the relative positional relation between the first part 22 and the second part 23 is changeable.

Since the first part 22 at which the insertion assist part 223 is provided can change the positional relation with the second part 23 at which the holding part 211 is integrally provided through the housing 21 or 21A in the pre-insertion state, the insertion assist part 223 can protrude on the skin S side from the second part 23, thereby forming the insertion complete state.

In the present embodiment, the through-hole 231 is provided at the second part 23, and the insertion assist part 223 is held through the through-hole 231.

Since the insertion assist part 223 is held through the through-hole 231, the through-hole 231 can guide the insertion assist part 223 when the first part 22 changes the relative positional relation with the second part 23, and accordingly, the insertion assist part 223 can stably protrude on the skin S side.

In the present embodiment, the through-hole 231 includes the large-diameter part 231a and the small-diameter part 231b, the large-diameter part 231a being provided on the micro needle array 4 side, the small-diameter part 231b being provided at a position further separated from the micro needle array 4 than the large-diameter part 231a, having a smaller diameter than the large-diameter part 231a, and slidably holding the insertion assist part 223, the protrusion 222 is provided at the insertion assist part 223, and the protrusion 222 passes through the small-diameter part 231b and generates notification sound when the magnitude of force toward the skin S exceeds the predetermined magnitude.

With the generation of the notification sound, it is possible to sense a state in which the magnitude of the force toward the skin S exceeds the predetermined magnitude and the micro needles 41 are reliably inserted into the skin. The generation of the notification sound serves as a trigger for separation of the applicators 2 and 2A from the skin S.

In the present embodiment, as described above with reference to FIGS. 5 to 9, the through-hole 231 may penetrate through the holding part 211, and the insertion assist part 223 may further protrude on the skin S side than the holding part 211 in the insertion complete state. Specifically, the hole 21Aa is provided at a position at the housing 21A on which the holding part 211 is provided, the position corresponding to the insertion assist part 223. The hole 21Aa and the through-hole 231 are connected to each other, and the through-hole 231 penetrates through the holding part 211.

Since the through-hole 231 penetrates through the holding part 211, the risk that the holding part 211 encumbers motion of the insertion assist part 223 is reduced. The holding part 211 maintains integration with the second part 23 and does not move toward the skin S side when the insertion assist part 223 further protrudes on the skin S side, and accordingly, the insertion assist part 223 presses the micro needle array 4 toward the skin S side so that the micro needle array 4 is separated from the holding part 211. Thus, it is possible to achieve both capability of holding the micro needle array 4 at the applicators 2 and 2A and capability of inserting the micro needle array 4 when force toward the skin S is applied.

The following describes an insertion test performed to check effects of such an aspect in which the micro needle array 4 is curved and pressed against the skin S and the micro needles 41 are reliably inserted into the skin S. The insertion test was performed as follows by using the micro needle array 4 provided with 120 micro needles 41.

(1) Blue pigment was applied to the micro needles 41, the micro needles 41 were set to the applicator 2, and the applicator 2 was placed on the skin S.

The first part 22 was pressed at 40 [N].

The applicator 2 was removed (the micro needle array 4 was bonded onto the skin S as illustrated in FIG. 5D)

The skin S was cut into 25 mm square together with the micro needle array 4 and the bonding tape 3.

Image capturing was performed with an X-ray µCT device.

The insertion depth was measured.

Results of the measurement are listed in Table 1. The protrusion length [mm] is the amount of protrusion from the second part 23 of the pressing rod 221 described above with reference to FIG. 4. The backing thickness [mm] is the thickness of a part at which the micro needles 41 are held at the micro needle array 4. The angle [°] is the angle of a top part of the micro needle array 4 in the curved state described above with reference to FIG. 5B. Thus, the angle is 90 [°] when the protrusion length is 0 [mm], and the angle decreases as the protrusion length increases.

TABLE 1

| Protrusion length [mm] | Backing thickness [mm] | Angle [°] | Insertion depth [µm] |
|---|---|---|---|
| 0 | 0.5 | 90 | 154 |
| 0.5 | 0.2 | 78 | 186 |
| 1.0 | 0.1 | 76 | 285 |
| 1.0 | 0.2 | - | 261 |
| 1.0 | 0.5 | - | 231 |
| 2.0 | 0.1 | 76 | - |
| 2.0 | 0.2 | - | 256 |

The shape of the leading end of the insertion assist part 223 is simplified in the above description with reference to FIGS. 1 to 9. An exemplary shape of the leading end of the insertion assist part 223 will be described below with reference to FIG. 10. An insertion assist part 223A exemplarily illustrated in FIG. 10 is formed by fabricating the leading end of a pressing rod 221A. The insertion assist part 223A is formed such that a central assist region 223Aa further protrudes on the skin S side than a periphery assist region 223Ab provided on the peripheral side of the central assist region 223Aa.

Since the insertion assist part 223A has a convex shape in which the periphery assist region 223Ab is further retracted from the skin S side than the central assist region 223Aa, a part of the micro needle array 4 pressed by the central assist region 223Aa as the insertion assist part 223A applies force toward the skin S side to the micro needle array 4 becomes closer to the skin S side and a part of the micro needle array 4 pressed by the periphery assist region 223Ab relatively becomes closer to the holding part 211. Thus, the micro needle array 4 has a curved shape, which increases capability of inserting the micro needle array and ensures capability of holding the micro needle array.

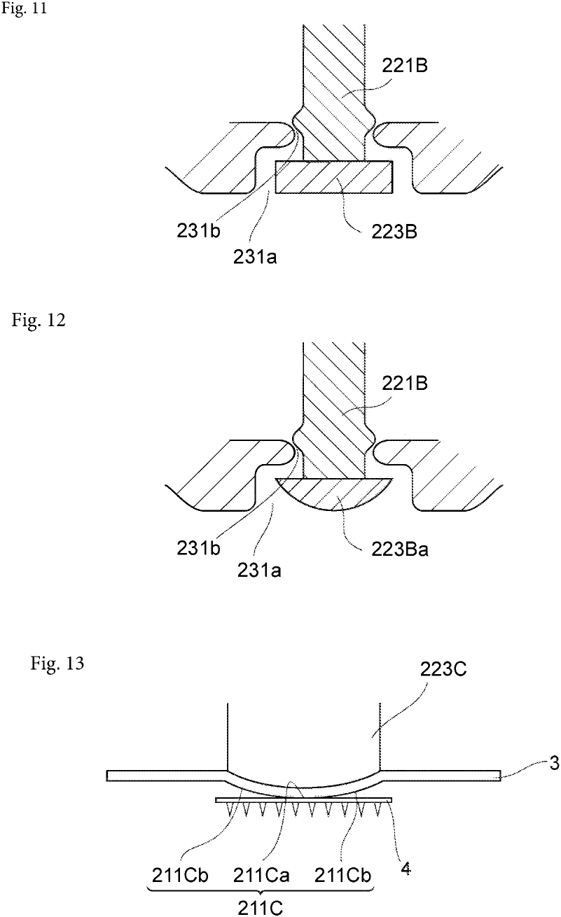

An insertion assist part 223B exemplarily illustrated in FIG. 11 is additionally attached to the leading end of a pressing rod 221B. The pressing rod 221B passes through the small-diameter part 231b. The insertion assist part 223B has a larger diameter than the small-diameter part 231b in accordance with the large-diameter part 231a.

An insertion assist part 223Ba exemplarily illustrated in FIG. 12 is additionally attached to the leading end of the pressing rod 221B. The insertion assist part 223Ba has a larger diameter than the small-diameter part 231b in accordance with the large-diameter part 231a. The insertion assist part 223Ba is formed such that a central part thereof protrudes on the skin S side.

When the insertion assist part is formed in a convex shape toward the skin S like the insertion assist parts 223A and 223Ba exemplarily illustrated in FIGS. 10 and 12, the insertion assist part and the holding part can be formed at the same position. An example in which the insertion assist part and the holding part are formed at the same position will be described below with reference to FIG. 13.

An insertion assist part 223C exemplarily illustrated in FIG. 13 has a convex shape in which the periphery assist region is further retracted from the skin S side than the central assist region. A holding part 211C is provided at the insertion assist part 223C. The holding part 211C is formed such that a central holding region 211Ca further protrudes on the skin S side than a peripheral holding region 211Cb provided on the peripheral side of the central holding region 211Ca.

Since the central array regions 42, 42A, and 42B (refer to FIGS. 16, 17, and 18) of the micro needle array 4 are held only in the central holding region 211Ca, the pre-insertion state in which the central array regions 42, 42A, and 42B do not further protrude on the skin S side than the peripheral array regions 43, 43A, and 43B (refer to FIGS. 16, 17, and 18) can be formed before the insertion complete state irrespective of the shape of the holding part 211C. When force is further applied while the holding part 211C is pressed against the skin S, the micro needle array 4 is deformed along the shape of the holding part 211C and the insertion complete state in which the central array regions 42, 42A, and 42B further protrude on the skin S side than the peripheral array regions 43, 43A, and 43B is formed.

Thus, the holding part 211C and the insertion assist part 223C do not need to be provided at different positions, but the insertion assist part 223C is formed when the central array regions 42, 42A, and 42B are held in the central holding region 211Ca. In this manner, the insertion assist part 223C can be formed with a simple contrivance of configuration, and accordingly, the configuration of the applicator can be simplified.

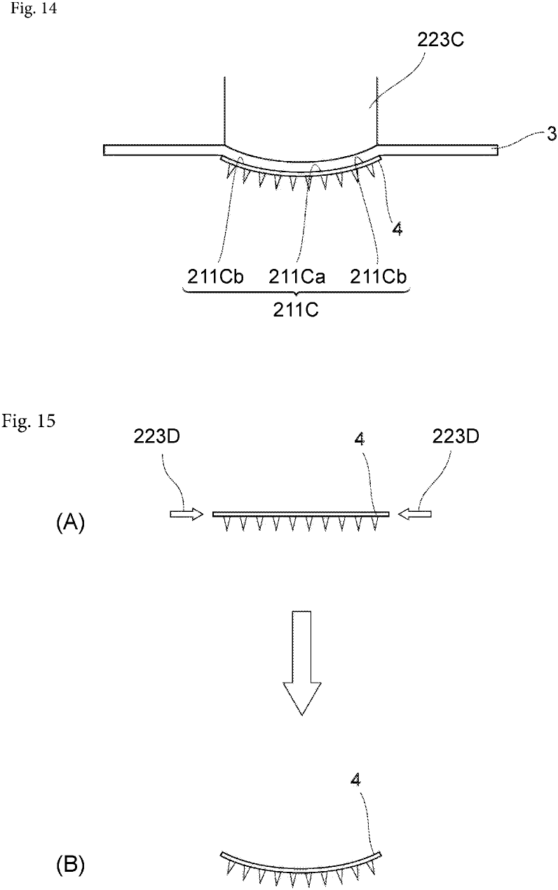

As illustrated in FIG. 14, the micro needle array 4 may be held in the central holding region 211Ca and the peripheral holding region 211Cb. When the micro needle array 4 is held in the central holding region 211Ca and the peripheral holding region 211Cb, a state in which the central array regions 42, 42A, and 42B further protrude on the skin S side along the shape of the holding part 211C than the peripheral array regions 43, 43A, and 43B can be formed and the insertion complete state can be reliably formed.

The above description is made on an example in which the insertion complete state is formed by applying force toward the skin S. An example in which the insertion complete state is formed by applying force along the skin S to the micro needle array 4 will be described below with reference to FIG. 15.

FIG. 15A illustrates the micro needle array 4 in the pre-insertion state. A state illustrated in FIG. 15B is obtained when an insertion assist part 223D applies force along the skin S to the micro needle array 4 as illustrated in FIG. 15A. FIG. 15B illustrates the micro needle array 4 in the insertion complete state. In this manner, the insertion assist part 223D forms the insertion complete state when receiving force that moves closer a pair of facing end parts of the micro needle array 4.

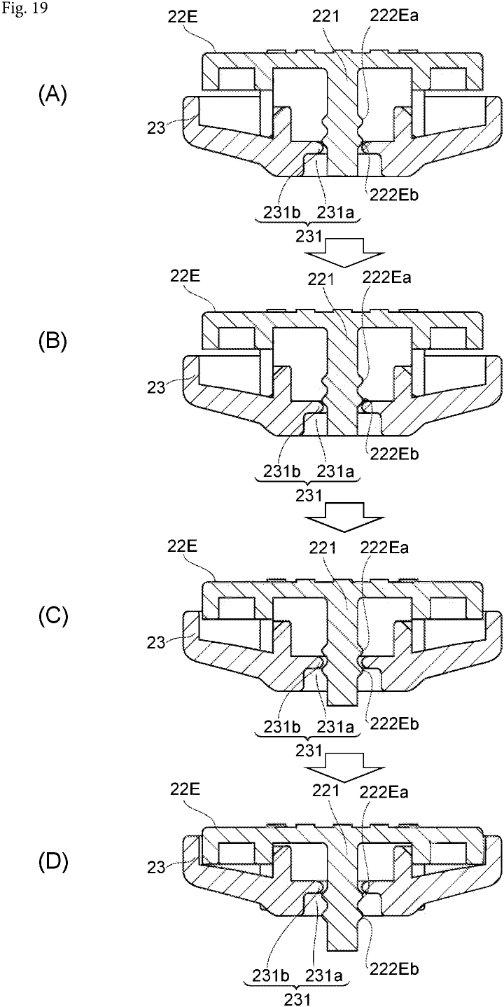

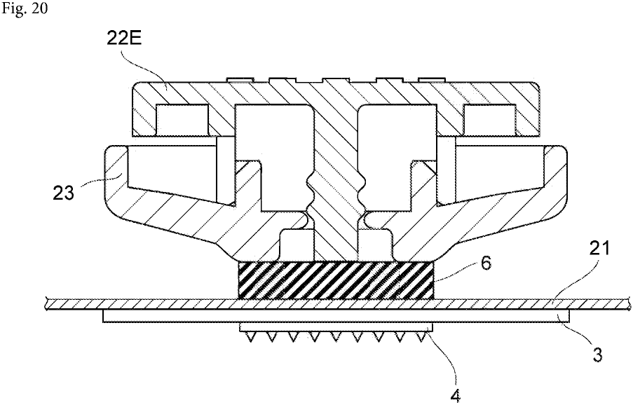

Although the single protrusion 222 is provided at the pressing rod 221 in the above description, a plurality of protrusions may be provided in the longitudinal direction of the pressing rod 221. Such an example will be described below with reference to FIG. 19.

In a first part 22E illustrated in FIGS. 19A, 19B, 19C, and 19D, a first protrusion 222Ea and a second protrusion 222Eb are provided at the pressing rod 221. FIG. 19A illustrates a state in which no force toward the skin is applied. The second protrusion 222Eb is provided on the skin side of the first protrusion 222Ea, and the first protrusion 222Ea is provided at a position further separated from the skin than the second protrusion 222Eb.

When force toward the skin is applied in the state illustrated in FIG. 19A, the insertion support part (refer to FIG. 3) applies force toward the skin to the micro needle array as illustrated in FIG. 19B, similarly to the state illustrated in FIG. 3.

A state illustrated in FIG. 19C is obtained when force toward the skin is further applied in the state illustrated in FIG. 19B and the magnitude of force applied to the first part 22E exceeds the predetermined magnitude. In the state illustrated in FIG. 19C, the second protrusion 222Eb passes through the small-diameter part 231b and the insertion assist part (refer to FIG. 4) of the pressing rod 221 protrudes on the skin side. Accordingly, the insertion assist part causes the central array region as part of the micro needle array to protrude on the skin side and insert the micro needles.

A state illustrated in FIG. 19D is obtained when force toward the skin is further applied in the state illustrated in FIG. 19C and the magnitude of force applied to the first part 22E exceeds the predetermined magnitude. In the state illustrated in FIG. 19D, the first protrusion 222Ea passes through the small-diameter part 231b and generates notification sound, thereby forming the insertion complete state.

With this configuration, the micro needle array is separated from the holding part (refer to FIG. 4) after the protrusion 222Eb as the first one of the protrusions passes through the small-diameter part 231b, and thus stress from the pressing rod 221 is likely to be applied only to the micro needle array so that insertion can be performed with less stress.

As illustrated in FIG. 20, an interposed part 6 made of a hard body such as resin or metal or made of an elastic body such as gel or sponge may be disposed between the second part 23 and the housing 21 so that the micro needle array 4 can be more easily put close to the skin. When the hard body is disposed, a through-hole may be provided at the center so that the central array region of the micro needle array 4 can be caused to protrude on the skin side with stress by the pressing rod 221.

The present embodiment may include a micro needle patch kit including the applicator 2 or 2A and a micro needle patch. In the micro needle patch, the micro needle array 4 is bonded to one surface of the bonding tape 3.

In the micro needle patch kit, the bonding tape 3 is provided with, in a region in which the insertion assist parts 223, 223A, 223B, 223Ba, 223C, and 223D contact a surface to which the micro needle array 4 is not bonded, a recessed part or surface fabrication that increases friction.

The present embodiment may include an insertion method of inserting the micro needle array 4 held by the applicator 2 or 2A into skin, the insertion method including inserting the micro needles 41 into the skin in a state in which the central array regions 42, 42A, and 42B of the micro needle array 4 further protrude on the skin side than the peripheral array regions 43, 43A, and 43B provided on the peripheral side of the micro needle array 4 than the central array regions 42, 42A, and 42B; and removing the micro needle array 4 from the applicator 2 or 2A.

The present embodiment is described above with reference to specific examples. However, the present disclosure is not limited to these specific examples. Those obtained by changing designing of the specific examples as appropriate by the skilled person in the art are included in the scope of the present disclosure as long as they have features of the present disclosure. Each element included in each above-described specific example and, for example, the disposition, condition, and shape thereof are not limited to those exemplarily illustrated but may be changed as appropriate. Combination of elements included in the above-described specific examples may be changed as appropriate without technological inconsistency.

1-16. (canceled)

17. An applicator for inserting a micro needle array into skin, the applicator comprising:
a holding part that holds the micro needle array;
an insertion assist part that forms an insertion complete state in which a central array region of the micro needle array further protrudes on the skin side than a peripheral array region provided closer to a peripheral side of the micro needle array than the central array region;
a first part at which the insertion assist part is provided; and
a second part at which the holding part is integrally provided at least in a pre-insertion state,
wherein a relative positional relation between the first part and the second part is changeable, and
wherein a through-hole is provided at the second part, and the insertion assist part is held through the through-hole.

18. The applicator according to claim 17, wherein the insertion assist part forms the pre-insertion state in which the central array region does not further protrude on the skin side than the peripheral array region before the insertion complete state.

19. The applicator according to claim 18, wherein, when receiving force toward the skin, the insertion assist part transitions from the pre-insertion state and forms the insertion complete state.

20. The applicator according to claim 17, wherein the holding part is provided on the peripheral array region.

21. The applicator according to claim 20,
wherein the through-hole includes a large-diameter part and a small-diameter part, the large-diameter part being provided on the micro needle array side, the small-diameter part being provided at a position farther separated from the micro needle array than the large-diameter part, having a smaller diameter than the large-diameter part, and slidably holding the insertion assist part,
wherein at least one protrusion is provided at the insertion assist part, and
wherein the protrusion passes through the small-diameter part when a magnitude of force toward the skin exceeds a predetermined magnitude.

22. The applicator according to claim 20,
wherein the through-hole penetrates through the holding part, and
wherein the insertion assist part further protrudes on the skin side than the holding part in the insertion complete state.

23. The applicator according to claim 20,
wherein the holding part is formed such that a central holding region further protrudes on the skin side than a peripheral holding region provided on the peripheral side of the central holding region, and
wherein the insertion assist part is formed when the central array region is held in the central holding region.

24. The applicator according to claim 20, wherein the insertion assist part is formed such that a central assist region further protrudes on the skin side than a periphery assist region provided on the peripheral side of the central assist region.

25. The applicator according to claim 20,
wherein the holding part is formed such that a central holding region further protrudes on the skin side than a peripheral holding region provided on the peripheral side of the central holding region, and
wherein the insertion assist part is formed when the micro needle array is held in the central holding region and the peripheral holding region.

26. The applicator according to claim 20, wherein the insertion assist part forms the insertion complete state when receiving force along the skin.

27. The applicator according to claim 26, wherein the insertion assist part forms the insertion complete state when receiving force that moves closer a pair of facing end parts of the micro needle array.

28. A micro needle patch kit, comprising:
the applicator according to claim 17; and
a micro needle patch,
wherein, in the micro needle patch, the micro needle array is bonded to one surface of a bonding tape.

29. The micro needle patch kit according to claim 28, wherein the bonding tape is provided with, in a region in which the insertion assist part contacts a surface to which the micro needle array is not bonded, a recessed part or surface fabrication that increases friction.

30. An insertion method of inserting a micro needle array held by an applicator into skin, the insertion method comprising:
inserting micro needles into the skin in a state in which a central array region of the micro needle array further protrudes on the skin side than a peripheral array region provided closer to a peripheral side of the micro needle array than the central array region; and
removing the micro needle array from the applicator.
